(19) United States
(12) Patent Application Publication
(10) Pub. No.: US 2021/0178306 A1
(43) Pub. Date: Jun. 17, 2021

(54) AROMATHERAPY MACHINE FOR AIR PURIFICATION

(71) Applicant: PUZHEN LIFE CO., LTD., Shatin (HK)

(72) Inventor: Andy LEE, Shatin (HK)

(21) Appl. No.: 16/833,295

(22) Filed: Mar. 27, 2020

(30) Foreign Application Priority Data
Dec. 17, 2019 (CN) 201922302702.6

(51) Int. Cl. B01D 46/00; A61L 9/14

(52) U.S. Cl. CPC B01D 46/0038; A61L 9/14; B01D 2279/50; B01D 46/0049; B01D 46/0043

(57) ABSTRACT

An aromatherapy machine for air purification, including: a housing, defining therein an air inlet and an air outlet; a fan, installed in the housing; a filter, configured for filtering and purifying air in the housing; and an aromatherapy device, arranged in the housing and configured to diffuse an essential oil for aromatherapy. The filter is installed in the housing, and the housing defines therein a mist outlet, configured to enable a diffused essential oil to diffuse to an outside of housing.

### CROSS-REFERENCE TO RELATED APPLICATION

The present application claims priority to Chinese Patent Application Ser. No. CN201922302702.6, filed on Dec. 17, 2019, the entire content of which is incorporated herein by reference in its entirety.

### BACKGROUND

### Technical Field

The present application relates to the field of air purification technology, and more particularly, relates to an aromatherapy machine for air purification.

### Description of Related Art

With the continuous improvement of people's living standards, more and more motorists generally put fragrance or aromatherapy machine in the car in order to avoid the deterioration of the air quality in the closed space of the car due to factors such as perspiration of the human body, emission of material gas in the car, or bacterial growth. Through release fragrance by aroma or diffusion and atomization of essential oil particles by aromatherapy machine, the air odor in the car is removed, and thus achieving the purpose of improving the air quality in the car. However, the aroma released by the fragrance or the atomized essential oil particles diffused by the aromatherapy machine usually has a lower purification efficiency of the air in the car, resulting in a poor improvement of the ambient air quality in the car and affecting the user experience.

### SUMMARY

The purpose of the present application is to provide an aromatherapy machine for air purification, so as to solve the problem that the aromatherapy machine in the related art has a low efficiency in purifying the air in the car and resulting in a poor effect of improving the ambient air quality in the car.

To achieve the above purpose, the technical scheme employed in the embodiment of the present application is to provide an aromatherapy machine for air purification, comprising:

a housing, defining therein an air inlet and an air outlet;

a fan, installed in the housing;

a filter, configured for filtering and purifying air in the housing; and

an aromatherapy device, arranged in the housing and configured to diffuse an essential oil for aromatherapy.

The filter is installed in the housing, and the housing defines therein a mist outlet, configured to enable a diffused essential oil to diffuse to an outside of housing.

In an embodiment, the fan is located at the air inlet and the filter is located at the air outlet; or the fan is located at the air outlet and the filter is located at the air inlet.

In an embodiment, a grid for dispersing an airflow is arranged in the air inlet.

In an embodiment, the grid comprises a grid frame and a plurality of grid plates. The plurality of grid plates are sequentially spaced inside the grid frame in parallel, and an air duct configured for enabling the airflow to pass therethrough is formed between every two adjacent grid plates.

In an embodiment, the plurality of the grid plates are arranged at equal intervals.

In an embodiment, the aromatherapy device comprises: an essential oil vial configured for storing the essential oil, and an atomization device configured for atomizing the essential oil from the essential oil vial and spraying out an atomized essential oil. The essential oil vial and the atomization device are arranged in the housing.

In an embodiment, the atomization device comprises: an air pump configured for providing an airflow, an atomization cover defining therein an atomization chamber, an air nozzle arranged in the atomization chamber, an air pipe connecting the air pump and the air nozzle, an oil suction pipe extending into the essential oil vial, and an oil nozzle connected to the oil suction pipe. The atomization cover is provided with a connection port configured for fitting connection with the essential oil vial, the connection port communicates with the atomization chamber, an outlet of the air nozzle is located at a position corresponding to an oil outlet of the oil nozzle, and the mist outlet communicates with the atomization chamber.

In an embodiment, an outer sidewall of an opening of the essential oil vial defines external threads, and an inner sidewall of the connection port defines internal threads configured to be fitted to the external threads.

In an embodiment, a bottom of the atomization chamber has a bevel extending obliquely to the connection port.

In an embodiment, the housing comprises: an air purification cylinder, and a pedestal supporting the air purification cylinder. The air inlet and the air outlet are located at two ends of the air purification cylinder, respectively. The fan and the filter are arranged inside the air purification cylinder.

The one or more technical schemes in the embodiments of the present application have at least one of the following technical effects:

The beneficial effect of the aromatherapy machine for air purification provided in the present application lies in that, compared with the prior art, the aromatherapy machine for air purification provided in the present application, the housing is provided with an aromatherapy device, and the essential oil diffuses by the aromatherapy device and the diffused essential oil is sprayed out of the mist outlet so that the aroma of the air in the car can be achieved. In addition, a fan and a filter are arranged in the housing, and a negative pressure airflow is generated in the housing by the fan. The air in the car is sucked in from the air inlet and filtered by the filter. The filtered and purified air is then discharged into the car from the air outlet to achieve circular purification function of air in the car. The fans and filters that filter and purify the air are installed in the housing to improve the purification efficiency of the air in the car. While the essential oil is diffused by aromatherapy device in the car, it can also quickly purify the air in the car to achieve a good aroma and purification effect.

### BRIEF DESCRIPTION OF THE DRAWINGS

In order to explain the technical schemes in the embodiments of the present disclosure more clearly, the drawings used in the embodiments will be briefly introduced below. Obviously, the drawings in the following description are only some embodiments of the present disclosure. For those of ordinary skill in the art, other drawings can be obtained based on these drawings without devoting creative labor.

FIG. 1 is a perspective view of an aromatherapy machine for air purification according to an embodiment of the present application;

FIG. 2 is a cross-sectional view of the aromatherapy machine;

FIG. 3 is an exploded view of the aromatherapy machine;

FIG. 4 is a partially enlarged view of an essential oil vial, an oil nozzle, and an air nozzle of the aromatherapy machine;

FIG. 5 is another partially enlarged view of a grid of the aromatherapy machine;

FIG. 6 is a perspective view of an ejection mechanism according to an embodiment of the present disclosure;

FIG. 7 is a cross-sectional view of an aromatherapy device and an ejection mechanism according to an embodiment of the present application;

FIG. 8 is an exploded view of the aromatherapy device and the ejection mechanism; and

FIG. 9 is a partially enlarged schematic view of a locking mechanism of the ejection mechanism.

The following reference numerals are used in the drawings:

1—Housing; 11—Air inlet; 12—Air outlet; 13—Air purification cylinder; 14—Pedestal; 15—Mist outlet; 16—Accommodation chamber;

2—Fan;

3—Filter;

4—Aromatherapy device; 41—Essential oil vial; 42—Atomization device; 421—Air pump; 422—Atomization cover; 423—Atomization chamber; 424—Air nozzle; 425—Oil nozzle; 426—Connection port; 427—Oil suction pipe;

5—Grid; 51—Grid frame; 52—Grid plate; 53—Air duct;

6—Ejection mechanism; 61—Outer sleeve; 611—Sliding groove; 62—Inner sleeve; 621—Slider; 622—Locking groove; 623—Limiting groove; 624—Slot; 63—Spring; 64—Locking mechanism; 641—Pull rod; 642—Hook; 65—Limiting spring arm; 66—Limiting block.

### DETAILED DESCRIPTION OF THE EMBODIMENTS

In order to make the purpose, technical scheme, and advantages of the present disclosure clearer, the present disclosure is further described in detail below with reference to the accompanying drawings and embodiments. It should be understood that the specific embodiments described herein are only used to explain the present disclosure and are not intended to limit the present disclosure.

It should be noted that when one component is called to be “fixed to” or “arranged on” another component, it may be directly on the other component or indirectly on the other component. When one component is referred to as being “connected to” another component, it can be directly connected to the other component or indirectly connected to the other component.

In addition, the terms “first” and “second” are used for descriptive purposes only and cannot be understood as indicating or implying relative importance or implicitly indicating the number of technical features indicated. Therefore, the features defined as “first” and “second” may explicitly or implicitly include one or more of the features. In the description of the present disclosure, the meaning of “a plurality of” is two or more, unless it is specifically defined otherwise.

In the description of the present application, it should be noted that the terms “installed”, “connected”, and “connecting” should be understood in a broad sense, unless explicitly stated and limited otherwise. For example, it may be fixedly connected or removably connected or connected in one piece; it can be mechanically connected or electrically connected; it can be directly connected or indirectly connected through an intermediate medium; it can be the internal connection of two elements or the interaction between two elements. For those of ordinary skill in the art, the specific meanings of the above terms in the present application can be understood according to specific situations.

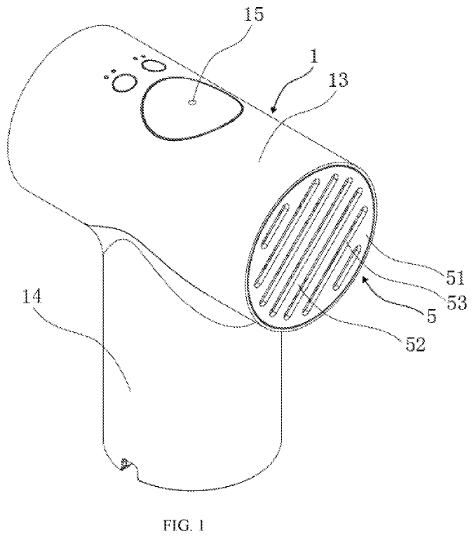

Referring to FIGS. 1, 2, and 3, an aromatherapy machine for air purification provided by the present application will now be described. The aromatherapy machine for air purification provided in the present application includes: a housing 1, a fan 2, a filter 3, and an aromatherapy device 4. The housing 1 defines therein an air inlet 11 and an air outlet 12, the fan 2 and the filter 3 are installed inside the housing 1, and the fan 2 is configured to generate a negative pressure airflow to allow the housing 1 to suck in air from outside, the filter 3 is configured to filter and purify the air in the housing 1. The aromatherapy device 4 is arranged in the housing 1, and the housing 1 defines a mist outlet 15, configured for enable a diffused essential oil to diffuse out of the housing 1. The Essential oil is diffused by aromatherapy device 4, and the diffused essential oil is sprayed out via the mist outlet 15, such that the air inside the car is incensed.

Compared with the prior art, an aromatherapy machine for air purification provided in the present application, in which, the aromatherapy device 4 is arranged in the housing 1, and the essential oil can be diffused by the aromatherapy device 4, and the diffused essential oil is sprayed out via the mist outlet 15, such that the air inside the car is incensed. In addition, the fan 2 and the filter 3 are arranged in housing 1. A negative pressure airflow is generated in the housing 1 by the fan 2, and the air in the car is sucked in from the air inlet 11 and filtered and purified by the filter 3. The filtered and purified air is then discharged into the car from the air outlet 12 to achieve circular purification function of the air in the car. Because the fan 2 and the filter 3 are installed in the housing 1 to filter and purify the air, the purification efficiency of the air in the car is improved. While the essential oil is diffused in the aromatherapy device 4, and the air in the car can be quickly purified, thereby achieving a good aroma and air purification effect.

Specifically, filter 3 may include, but not limited to, an electrostatic electret filter with low wind resistance, high efficiency, and high dust holding capacity, an activated carbon filter with a high volume of pore structure and a large surface area, or a high efficient particulate air filter (HEPA filter).

In one embodiment, as a specific implementation of the aromatherapy machine for air purification provided in the present application, the fan 2 is located at the air inlet 11, and filter 3 is located at air outlet 12. In this embodiment, by arranging the fan 2 at the air inlet 11 and by arranging the filter 3 at the air outlet 12, it is beneficial to increase the air pressure of the air purification cylinder 13 and to improve the efficiency of air filtration and purification.

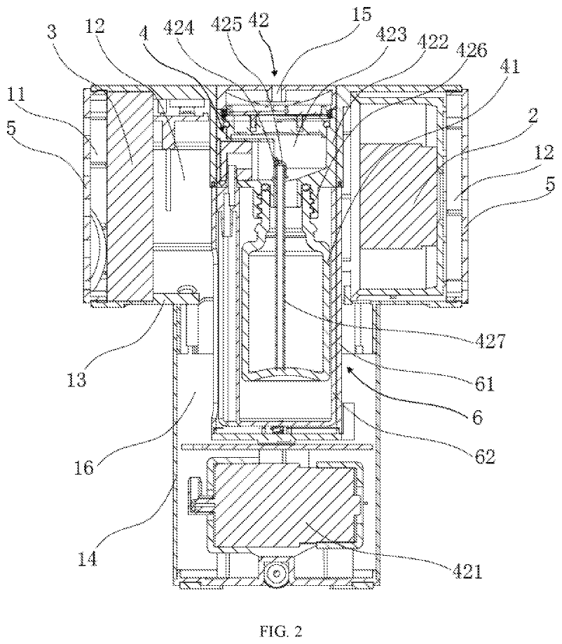

According to an embodiment, referring to FIGS. 2-4, as a specific implementation of the aromatherapy machine for air purification provided in the present application, the fan 2 is located at the air outlet 12 and the filter 3 is located at the air inlet 11. In the embodiment, by arranging the fan 2 at the air outlet 12 and by arranging the filter 3 at the air inlet 11, it is beneficial to increase the air pressure of the air purification cylinder 13 and to improve the efficiency of air filtration and purification.

According to an embodiment, referring to FIGS. 2-4, as a specific implementation of the aromatherapy machine for air purification provided in the present application, a grid 5 is arranged within the air inlet 11 for dispersing airflow. In this embodiment, the grid 5 is arranged in the air inlet 11, and the airflow is dispersed by the grid 5 to prevent the airflow from flowing too concentratedly to the air purification cylinder 13, which has a strong impact on the filter 3 of the air purification cylinder 13 and affects the filtering effect.

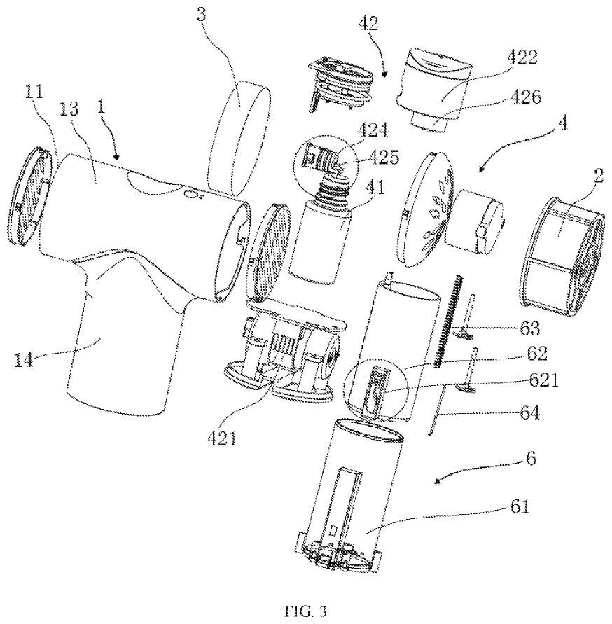

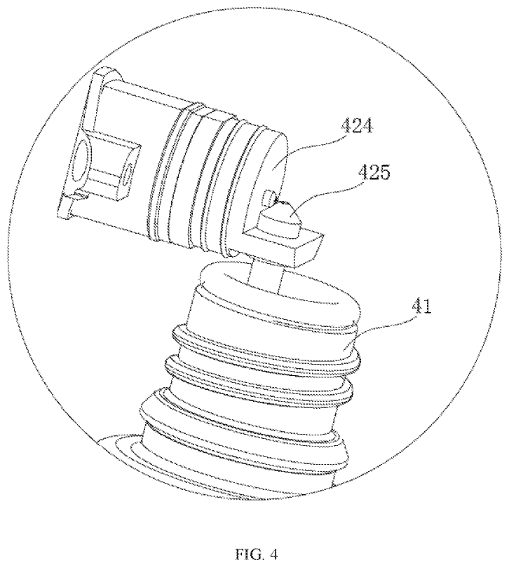

According to an embodiment, referring to FIGS. 3-4, as a specific implementation of the aromatherapy machine for air purification provided in the present application, the air outlet 12 may further be provided therein with a grid 5 for adjusting the flow direction of the airflow. The grid 5 is capable of adjusting the direction of the airflow, thereby changing the blowing direction of air outlet 12 of the air purification cylinder 13, so that the filtered and purified air is quickly discharged to all corner spaces in the car, which is beneficial to improve the efficiency of the ambient air quality in the car.

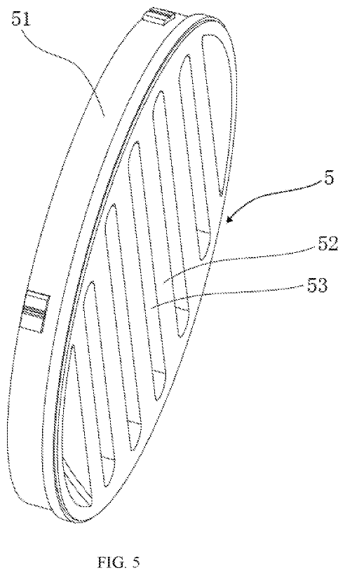

According to an embodiment, referring to FIG. 5, as a specific implementation of an aromatherapy machine for air purification provided in the present application, the grid 5 includes a grid frame 51 and a plurality of grid plates 52, and the plurality of grid plates 52 are sequentially spaced in parallel inside the grid frame 51. An air duct 53 configured for the airflow to pass therethrough is formed between every two adjacent grid plates 52.

In this embodiment, the grid 5 includes a grid frame 51 and a plurality of grid plates 52 which are sequentially spaced in parallel inside the grid frame 51. Each grid plate 52 and an adjacent grid plate 52 form an air duct 53, respectively, for changing the blowing direction, and the airflow discharged through the air outlet 12 effectively changes the blowing direction and adjusts the blowing flow rate under the action of air duct 53, so that the filtered and purified air is quickly discharged to all corner spaces in the car.

According to an embodiment, referring to FIG. 5, as a specific implementation of the aromatherapy machine for air purification provided in the present application, the plurality of grid plates 52 are arranged at equal intervals. In this embodiment, a plurality of grid plates 52 are arranged at equal intervals, so that the airflow velocity of the air blown out by the grid is uniform, and a good exhaust effect is achieved, thereby preventing turbulent airflow due to uneven exhaust flow velocity, which would otherwise produce noise and reduce user experience.

According to embodiment, referring to FIGS. 2-4, as a specific implementation of the aromatherapy machine for air purification provided in the present application, the aromatherapy device 4 includes: an essential oil vial 41 configured for storing the essential oil, and an atomization device 42 configured for atomizing the essential oil from the essential oil vial 41 and spraying out an atomized essential oil, and the essential oil vial 41 and the atomization device 42 are arranged in the housing 1.

In this embodiment, the aromatherapy device 4 includes an essential oil vial 41 and an atomization device 42 arranged in the housing 1. The essential oil can be stored in the essential oil vial 41 in advance, and the essential oil in the essential oil vial 41 can be atomized into mist-like essential oil particles by the atomization device 42. The mist-like essential oil particles are diffused into the air in the car through the mist outlet 15 of the housing 1, and the air in the car can be incensed.

Accordingly to an embodiment, referring to FIGS. 2-4, as a specific implementation of the aromatherapy machine for air purification provided in the present application, the atomization device 42 includes: an air pump 421 configured for providing an airflow, an atomization cover 422 defining therein an atomization chamber 423, an air nozzle 424 arranged in the atomization chamber 423, an air pipe (not shown in the figure) connecting the air pump 421 and the air nozzle 424, an oil suction pipe 427 extending into the essential oil vial 41, and an oil nozzle 425 connected to the oil suction pipe 427. The atomization cover 422 is provided with a connection port 426 configured for fitting connection with the essential oil vial 41. The connection port 426 communicates with the atomization chamber 423. An outlet of the air nozzle 424 is located at a position corresponding to an oil outlet of the oil nozzle 425. The atomization chamber 423 communicates with the mist outlet 15.

In this embodiment, the atomization cover 422 defines therein the atomization chamber 423. The atomization cover 422 defines the mist outlet 15 communicating with the atomization chamber 423. A lower end of the atomization cover 422 is provided with a connection port 426. A vial mouth of the essential oil vial 41 is connected to the atomization cover 422 through the connection port 426. The atomization cover 422 is installed on the housing 1, and the air pump 421 is installed in the housing. The housing 1 supports and protects the air pump 421. One end of the air pipe is connected to the air pump 421, and the other end of the air pipe is connected to the air nozzle 424. The airflow provided by the air pump 421 is transmitted to the air nozzle 424 through the air pipe, and is ejected from the air nozzle 424. The oil outlet of the oil nozzle 425 is located in the atomization chamber 423, the outlet of the air nozzle 424 is located at a position corresponding to the oil nozzle 425, and the oil nozzle 425 is located at a position corresponding to the connection port 426, so that when the connection port 426 is connected to the essential oil vial 41, the oil nozzle 425 can extract the essential oil from the essential oil vial 41 through the oil suction pipe 427, and the upper end of the oil nozzle 425 extends into the atomization chamber 423, and the outlet of air nozzle 424 is located at the upper end of oil nozzle 425; thus, when air pump 421 generates high-pressure gas and the high-pressure gas is released from air nozzle 424, a negative pressure is formed at the upper end of the oil nozzle 425 to extract the essential oil from the essential oil vial 41 through the oil suction pipe 427. The extracted essential oil is ejected from the oil outlet of the oil nozzle 425. The liquid droplets will be blown away by the high-speed airflow sprayed from the air nozzle 424 and be atomized to form a mixed airflow, and at the same time, the pressure in the atomization chamber 423 will be increased; and as the connection port 426 is connected to the essential oil vial 41, the airflow in the atomization chamber 423 will be sprayed out of the mist outlet 15 to incense the air in the car and improve the air quality inside the car

According to an embodiment, referring to FIGS. 2-4, as a specific implementation of the aromatherapy machine for air purification provided in the present application, an outer sidewall of an opening of the essential oil vial 41 defines external threads, and an inner sidewall of the connection port 426 defines internal threads fitted to the external threads.

In this embodiment, the external threads are arranged on the outer sidewall of the opening of the essential oil vial 41, and the internal threads are arranged on the inner sidewall of the connection port 426. By matching the external threads with the internal threads, it is convenient and fast to connect the essential oil vial 41 and the connection port 426, and the sealing of the connection between the essential oil vial 41 and the connection port 426 can be enhanced.

According to an embodiment, referring to FIG. 2, as a specific implementation of the aromatherapy machine for air purification provided in the present application, a bottom of the atomization chamber 423 has a bevel extending obliquely to the connection port 426.

In this embodiment, the bottom of the atomization chamber 423 has a bevel that extends obliquely to the connection port 426. Through the guidance of the bevel, large particles of essential oil droplets are guided back to the essential oil vial 41 to avoid affecting the essential oil atomization effect and reduce essential oil waste.

According to an embodiment, referring to FIGS. 1-3, as a specific implementation of the aromatherapy machine for air purification provided in this application, the housing 1 includes: an air purification cylinder 13, and a pedestal 14 supporting the air purification cylinder 13. The air inlet 11 and the air outlet 12 are located at two ends of the air purification cylinder 13, respectively and the fan 2 and the filter 3 are arranged inside the air purification cylinder 13.

In this embodiment, the housing 1 includes: the air purification cylinder 13 extending axially and horizontally, and the pedestal 14 supporting the air purification cylinder 13. The air inlet 11 and the air outlet 12 are located at the two ends of the air purification cylinder 13, respectively. The fan 2 and the filter 3 are arranged inside the air purification cylinder 13. When in use, a negative pressure airflow is generated in the air purification cylinder 13 through the fan 2, and the air in the car is sucked in from the air inlet 11 at one end of the air purification cylinder 13 and filtered and purified by the filter 3. The filtered and purified air is then discharged from the air outlet 12 at the other end of the air purification cylinder 13 into the car, realizing the circular purification function of the air in the car. As the air inlet 11 and the air outlet 12 are arranged at the two ends of the air purification cylinder 13, respectively, the wind resistance is reduced, the fast convection of the airflow is easily realized, the purification efficiency of the air in the car is improved, and the air quality in the car environment is improved.

According to an embodiment, referring to FIG. 2, as a specific implementation of the aromatherapy machine for air purification provided in the present application, the pedestal 14 defines therein an accommodation chamber 16 which is in communication with the air purification cylinder 13, and the air pump 421 is arranged in the accommodation chamber 16. The atomization cover 422 is arranged in the air purification cylinder 13, and the essential oil vial 41 is located between the air pump 421 and the atomization cover 422. In this embodiment, an accommodation chamber 16 communicating with the air purification cylinder 13 is arranged on the pedestal 14 to put the air pump 421 in the accommodation chamber 16, the atomization cover 422 is arranged in the air purification cylinder 13, and the essential oil vial 41 is located between the air pump 421 and the atomization cover 422. The structural layout of the aromatherapy machine used for air purification is compact and reasonable, which is beneficial to reduce the volume of the aromatherapy machine used for air purification, taking up less space and facilitating the placement in a small space in the car.

According to an embodiment, referring to FIGS. 2-3, as a specific implementation of the aromatherapy machine for air purification provided in the present application, an end of the oil nozzle 425 away from the oil outlet is provided with a connection sleeve. The oil suction pipe 427 is inserted into the oil nozzle 425 detachably, and the oil suction pipe 427 is arranged in the essential oil vial 41.

In this embodiment, a connection sleeve is arranged at an end of the oil nozzle 425 away from the oil outlet, and an oil suction pipe 427 is detachably inserted in the connection sleeve. The oil suction pipe 427 is connected to the lower end of the oil nozzle 425. The essential oil in the essential oil vial 41 is sucked into the oil nozzle 425 by the negative pressure. In addition, the oil suction pipe 427 is detachably inserted into the connection sleeve of the oil nozzle 425, and the oil suction pipe 427 of different lengths can be replaced according to different essential oil vial 41 to improve the adaptability.

According to an embodiment, referring to FIGS. 2-4, as a specific implementation of the aromatherapy machine for air purification provided in the present application, the housing 1 is provided therein with an accommodation space for accommodating and positioning an essential oil vial 41 to accommodate and position the essential oil vial 41 in the accommodation space, in order to facilitate the installation of the essential oil vial 41, and to enhance the stability of the essential oil bottle 41 installation.

According to an embodiment, referring to FIGS. 3-4, as a specific implementation of the aromatherapy machine for air purification provided in the present application, an axial direction of the outlet of the air nozzle 424 is directed to a top of an upper side wall of the oil nozzle 425. Therefore, when the air is sprayed from the outlet of the air nozzle 424, the air can cover the upper end of the oil nozzle 425, to better form a vacuum at the upper end of the oil nozzle 425. At the same time, the top of the upper side wall of the oil nozzle 425 can change the flow direction of the air nozzle 424, and further, the essential oil droplets extracted by the oil nozzle 425 can be better atomized.

According to an embodiment, referring to FIGS. 3-4, as a specific implementation of the aromatherapy machine for air purification provided in the present application, the axial direction of the outlet of the air nozzle 424 is inclined upwardly to the top of side wall of the upper end of the oil nozzle 425. This structure can prevent the airflow sprayed out of the air nozzle 424 from being blown into the pipe outlet of the upper end of the oil nozzle 425, so that the essential oil can be better extracted, and the extracted essential oil can be obliquely and upwardly blown out, which can better atomize the essential oil.

According to an embodiment, referring to FIGS. 3-4, as a specific implementation of the aromatherapy machine for air purification provided in the present application, the side wall of the upper end of the oil nozzle 425 is conical, so that the side wall of the upper end of the oil nozzle 425 can guide the airflow ejected from the air nozzle 424 upwardly, thus the airflow can better atomize the essential oil extracted by the oil nozzle 425. In other embodiments, the side wall of the upper end of the oil nozzle 425 may also be arc-shaped convex upward.

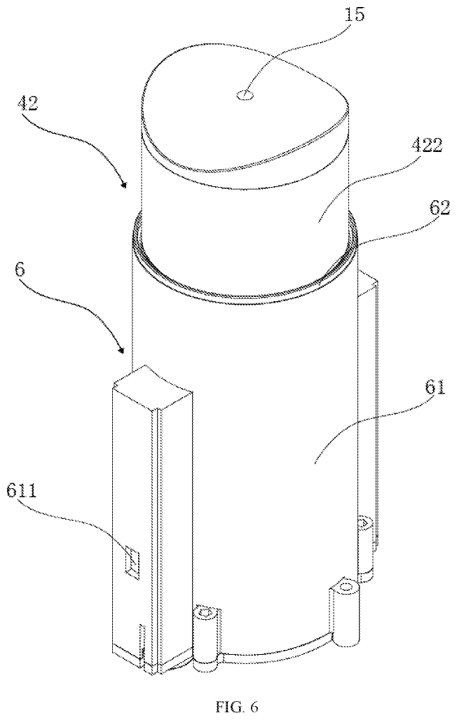

According to an embodiment, referring to FIGS. 2, 6, and 7, as a specific implementation of the aromatherapy machine for air purification provided in the present application, the housing 1 defines an accommodation slot fitted with an atomization device 42. An accommodation slot is provided with an ejection mechanism 6 for ejecting the atomization device 42.

In this embodiment, an accommodation slot is defined in housing 1. The accommodation slot is used to accommodate the atomization device. When in use, the atomization device can be built in the accommodation slot to hide the atomization device, which achieves detachable connection and avoids the atomization device damage. In addition, an ejection mechanism 6 for ejecting an atomization device is arranged in the accommodating slot. When the atomization device is damaged, the atomization device can be ejected from the accommodating slot by the ejection mechanism 6, and the atomization device can be conveniently removed from the accommodating slot. Easy maintenance and replacement of the atomization device could extend the life of the aromatherapy device 4 and reduce the cost of the aromatherapy device 4 utilization.

According to an embodiment, referring to FIGS. 2, 6, and 7, as a specific implementation of the aromatherapy machine for air purification provided in this application, the ejection mechanism 6 includes an outer sleeve 61 fixed in the accommodation slot, an inner sleeve 62 for accommodating the atomization device 42, a spring 63 for elastically pushing the inner sleeve 62 outward, and a locking mechanism 64 for locking the inner sleeve 62 inside the outer sleeve 61. The inner sleeve 62 is slidably installed in the outer sleeve 61, a spring 63 is arranged in the outer sleeve 61, a locking mechanism 64 is connected to the outer sleeve 61, and an inner sleeve 62 is connected to the atomization device 42.

In this embodiment, the ejection mechanism 6 includes: an outer sleeve 61, an inner sleeve 62, a spring 63, and a locking mechanism 64. The outer sleeve 61 is fixedly connected to an accommodation slot, and the inner sleeve 62 is slidably installed in the outer sleeve 61. The spring 63 is arranged in the outer sleeve 61, and the inner sleeve 62 is elastically pushed from the bottom of the outer sleeve 61 toward the top of the outer sleeve 61 by the spring 63. When the atomization device is arranged in the accommodation slot, the atomization device is arranged and connected to the inner sleeve 62, and then press the atomization device along the inner sleeve 62 (down) with force, to overcome the elastic pushing force of the spring 63 (up), the atomization device following with the inner sleeve 62 slides to the inside of the outer sleeve 61 along the axial direction of the outer sleeve 61, and the inner sleeve 62 is locked in the outer sleeve 61 with the locking mechanism 64, so that the atomization device is built in and hidden in the housing 1 for use. In addition, by hiding the atomization device in housing 1, the atomization device can be protected by housing 1 to avoid damage to the atomization device. At the same time, the atomization device is built in housing 1, which reduces the volume of the entire aromatherapy device 4, which is convenient for placing the aromatherapy device 4 in a small area. When it is necessary to repair or replace the atomization device, the locking mechanism 64 only need to unlock the inner sleeve 62, the inner sleeve 62 is lifted from the bottom of the outer sleeve 61 toward the top of the outer sleeve 61 by the spring 63 under the elastic pushing effect of the spring 63 (upward), so that the atomization device following with the inner sleeve 62 slides to the outside of the outer sleeve 61 along the axial direction of outer sleeve 61, and then ejects from the accommodation slot, and the atomization device can be easily taken out from the accommodation slot, and is easy to repair and replace.

In another embodiment, as a specific implementation of the aromatherapy device 4 provided in the present application, the ejection mechanism 6 may further include: an outer sleeve 61 fixed in the accommodation slot, an inner sleeve 62 used to accommodate the atomization device and fitted to connect with the atomization device, a spring 63 that elastically pushes the inner sleeve 62 outward, and a locking mechanism 64 for locking the inner sleeve 62 inside the outer sleeve 61. The inner sleeve 62 is slidably installed in the outer sleeve 61, the locking mechanism 64 is a buckle arranged on the outer sleeve 61, and an inner sleeve 62 defines a groove that is fitted with the buckle. The inner sleeve 62 is locked to the outer sleeve 61 by an interference fit buckle mechanism. The atomization device can be conveniently built into the housing 1 for use, or the atomization device will be taken out from the accommodation slot, which is convenient for maintenance and replacement of the atomization device. It can be understand that an external thread may be defined on the outer wall of the inner sleeve 62, and an internal thread may be defined on the inner wall of the outer sleeve 61. The inner sleeve 62 is locked to the outer sleeve by connecting the inner sleeve 62 with the external and internal threads, the atomization device can be conveniently built into housing 1 for use, or the atomization device will be taken out from the accommodation slot, which is convenient for maintenance and replacement of the atomization device.

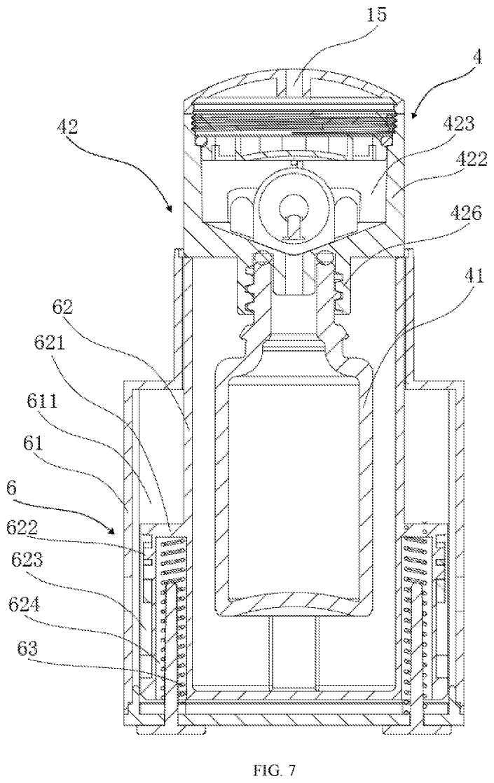

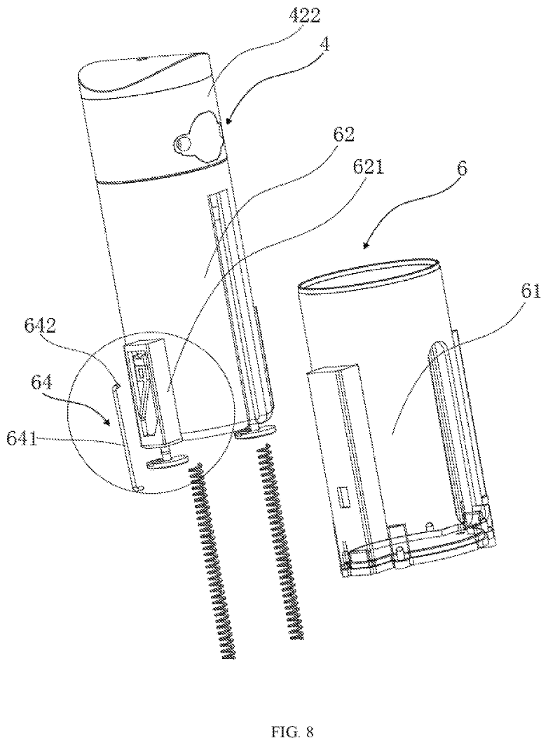

According to an embodiment, referring to FIGS. 7 and 8, as a specific implementation of the aromatherapy machine for air purification provided in the present application, an inner side wall of the outer sleeve 61 is recessed to form a sliding groove 611. A slider 621 fitting with the sliding groove 611 is protruded on an outer side wall of the inner sleeve 62.

In this embodiment, the inner side wall of the outer sleeve 61 is recessed to form the sliding groove 611, and the slider 621 is correspondingly convexly formed on the outer side wall of the inner sleeve 62. By the combination of the slider 621 and the slide groove 611, the inner sleeve 62 is installed by sliding in the outer sleeve 61 to enhance the stability of the movement of the inner sleeve 62 along the outer sleeve 61 axial direction, which is beneficial to the rapid and stable installation or detachment of the atomization device.

According to an embodiment, referring FIGS. 3, 7, and 8, as a specific implementation of the aromatherapy machine for air purification provided in the present application, the locking mechanism 64 includes: a pull rod 641 arranged in the slide groove 611, and a hook 642 bent from one end of the pull rod 641 toward the slider 621; and the other end of the pull rod 641 is fixed on the outer sleeve 61. The slider 621 is provided with a locking groove 622 for engaging with the hook 642 to lock or unlock.

In this embodiment, the locking mechanism 64 includes the pull rod 641 and the hook 642 bent from one end of the pull rod 641 toward the slider 621. The hook is located at the sliding groove 611, and the end of the pull rod 641 away from the hook 642 is fixed to the outer sleeve 61. When the inner sleeve 62 slides to the bottom of the outer sleeve 61, the locking groove 622 is locked by the hook 642 to lock the inner sleeve 62 in the outer sleeve 61. The atomization device following with the inner sleeve 62 slides to the inside of the outer sleeve 61 along the outer sleeve 61 axial direction, so that the atomization device is built in and hidden in housing 1 for use. When the atomization device needs to be repaired or replaced, the hook 642 only needs to be released from the locking groove 622 and the inner sleeve 62 is unlocked, the spring 63 (upward) elastically pushes the inner portion by the spring 63. The inner sleeve 62 is elastically pushed from the bottom of the outer sleeve 61 toward the top of the outer sleeve 61, so that the atomization device following with the inner sleeve 62 slides to the outside of the outer sleeve 61 along the outer sleeve 61 axial direction, and then the accommodation slot is ejected. So that atomization device could be easily removed from the accommodation slot for maintenance and replacement of the atomization device.

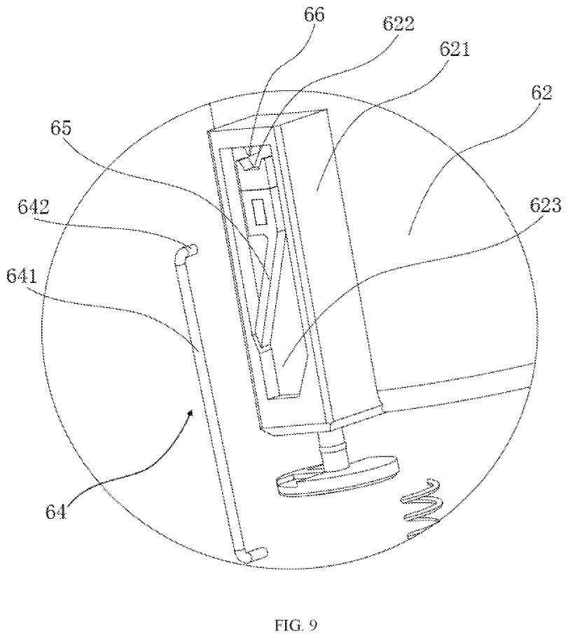

According to an embodiment, referring to FIGS. 4, 8, and 9, as a specific implementation of the aromatherapy machine for air purification provided in the present application, a slider 621 is provided with a limiting groove 623, configured for guiding the hook 642 to move in or out of the of the locking groove 622, the locking groove 622 is located inside the limiting groove 623.

In this embodiment, a slider 621 is provided with a limiting groove 623, configured for guiding the hook 642 to move in or out of the locking groove 622, the locking groove 622 is located inside the limiting groove 623. The movement path of the hook 642 is guided by the limiting groove 623, so that the hook 642 can smoothly move in or out of the locking groove 622, and the inner sleeve 62 can be locked or unlocked and the smoothness and stability of the hook 642 to be moved in or out of the locking groove 622 could be enhanced, which facilitates the installation and detachment of the atomization device.

According to an embodiment, referring to FIGS. 4, 8, and 9, as a specific implementation of the aromatherapy machine for air purification provided in the present application, the limiting groove 623 is provided with a limiting spring arm 65 configured for stopping the hook 642 and defining the movement path of the hook 642 in the limiting groove 623.

In this embodiment, a limiting spring arm 65 is arranged in the limiting groove 623, and the hook 642 can be stopped by the limiting spring arm 65 to limit the movement path of the hook 642 in the limiting groove 623, thereby further making the hook 642 capable of moving in or out of the locking groove 622 quickly and smoothly, to achieve the locking or unlocking of the inner sleeve 62 and to enhance the smoothness and stability of the hook 642 moving in or out of the locking groove 622, thus facilitating the installation and detachment of the atomization device.

According to an embodiment, referring to FIGS. 4, 8, and 9, as a specific implementation of the aromatherapy machine for air purification provided in the present application, an inner wall of the limiting groove 623 is convex to form a limiting block 66 for stopping the hook 642 and defining a movement path of the hook 642, and the position of the limiting block 66 correspond to the opening position of the locking groove 622.

In this embodiment, a limiting block 66 is protruded on the inner side wall of the limiting groove 623, and when the hook 642 moves out of the locking groove 622, the limiting block 66 is used to stop hook 642 coordinately, the movement path of the hook 642 is limited, so that the hook 642 can move out of the lock groove 622 quickly and stably, and the inner sleeve 62 can be quickly unlocked. The efficiency of the installation and detachment of the atomization device can be improved, and the stability of installation and detachment of the atomization device can be enhanced.

According to an embodiment, referring to FIG. 7, as a specific implementation of the aromatherapy machine for air purification provided in the present application, the slider 621 is provided with a slot 624 for inserting the top end of the spring 63, and the slot 624 extends along an axial direction of the inner sleeve 62, and a bottom end of the spring 63 is connected to the inner bottom surface of the outer sleeve 61.

In this embodiment, the slider 621 is provided with a slot 624 for inserting the top end of the spring 63, and the slot 624 extends along the axial direction of the inner sleeve 62, and the top end of the spring 63 is arranged in the slot 624. The bottom end of the spring 63 is connected to the inner bottom surface of the outer sleeve 61, so that the spring 63 is defined by the slot 624, which enhances the stability of the spring 63 to push the inner sleeve 62 elastically.

The above description is only the preferred embodiments of this application, and is not intended to limit this application. Any modification, equivalent replacement, and improvement made within the spirit and principle of this application shall be included in the protection range of this application.

What is claimed is:

1. An aromatherapy machine for air purification, comprising:
a housing, defining therein an air inlet and an air outlet;
a fan, installed in the housing;
a filter, configured for filtering and purifying air in the housing; and
an aromatherapy device, arranged in the housing and configured to diffuse an essential oil for aromatherapy;
wherein the filter is installed in the housing, and the housing defines therein a mist outlet, configured to enable a diffused essential oil to diffuse to an outside of housing.

2. The aromatherapy machine for air purification according to claim 1, wherein the fan is located at the air inlet and the filter is located at the air outlet; or the fan is located at the air outlet and the filter is located at the air inlet.

3. The aromatherapy machine for air purification according to claim 1, wherein a grid for dispersing an airflow is arranged in the air inlet.

4. The aromatherapy machine for air purification according to claim 3, wherein the grid comprises a grid frame and a plurality of grid plates; the plurality of grid plates are sequentially spaced inside the grid frame in parallel; and an air duct configured for enabling the airflow to pass therethrough is formed between every two adjacent grid plates.

5. The aromatherapy machine for air purification according to claim 4, wherein the plurality of the grid plates are arranged at equal intervals.

6. The aromatherapy machine for air purification according to claim 1, wherein the aromatherapy device comprises: an essential oil vial configured for storing the essential oil, and an atomization device configured for atomizing the essential oil from the essential oil vial and spraying out an atomized essential oil; and the essential oil vial and the atomization device are arranged in the housing.

7. The aromatherapy machine for air purification according to claim 6, wherein
the atomization device comprises: an air pump configured for providing an airflow, an atomization cover defining therein an atomization chamber, an air nozzle arranged in the atomization chamber, an air pipe connecting the air pump and the air nozzle, an oil suction pipe extending into the essential oil vial, and an oil nozzle connected to the oil suction pipe; and
the atomization cover is provided with a connection port configured for fitting connection with the essential oil vial, the connection port communicates with the atomization chamber, an outlet of the air nozzle is located at a position corresponding to an oil outlet of the oil nozzle, and the mist outlet communicates with the atomization chamber.

8. The aromatherapy machine for air purification according to claim 7, wherein an outer sidewall of an opening of the essential oil vial defines external threads, and an inner sidewall of the connection port defines internal threads configured to be fitted to the external threads.

9. The aromatherapy machine for air purification according to claim 7, wherein a bottom of the atomization chamber has a bevel extending obliquely to the connection port.

10. The aromatherapy machine for air purification according to claim 1, wherein the housing comprises: an air purification cylinder, and a pedestal supporting the air purification cylinder; the air inlet and the air outlet are located at two ends of the air purification cylinder, respectively; and the fan and the filter are arranged inside the air purification cylinder.
